(19) United States
(12) Patent Application Publication
(10) Pub. No.: US 2006/0136058 A1
(43) Pub. Date: Jun. 22, 2006

(54) Patient specific anatomically correct implants to repair or replace hard or soft tissue

(76) Inventor: William Pietrzak, Westwood, MA (US)

Correspondence Address: ARENDT & ASSOCIATES INTELLECTUAL PROPERTY GROUP, 1740 MASSACHUSETTS AVENUE, BOXBOROUGH, MA, 01719-2209, US

(21) Appl. No.: 11/312,950

(22) Filed: Dec. 19, 2005

Related U.S. Application Data
(60) Provisional application No. 60/637,320, filed on Dec. 17, 2004.

(51) Int. Cl. A61F 2/08; A61F 2/30; G06F 19/00

(52) U.S. Cl. 623/13.14; 623/901; 700/119; 623/18.11

(57) ABSTRACT

The present invention relates to a system of preparing a one of a kind artificial or biological implant to repair or replace a bone or a tissue part in a living individual, the system comprising: pre-operatively constructing an anatomically correct, affected area 7-unique scan implants geometry, wherein the implants geometry is unknown before the acquisition of the individual's data; and using a process derived from rapid prototyping to construct a geometrically and anatomically correct duplication of the functional attributes of the bone or tissue part to be replaced from the dataset created by scans of the recipients scans. This system can also be used to modify, pre-operatively, existing FDA approved implants.

### RELATED APPLICATION

This application claims the benefit of U.S. Provisional Application No. 60/637,320 filed on Dec. 17, 2004, the teachings of which are incorporated herein by reference in their entirety.

### BACKGROUND OF THE INVENTION

Although current joint repair and joint replacement technology allows for computerized tomography (CT) or magnetic resonance imaging (MRI) scans to be voxelized, that is, converted to computer generated 3D models, for simulation in order to allow the surgical team to select the optimal size and orientation of an implant, the implant and the living tissue to which it attaches must be modified in the operating room to obtain the best possible fit and Range of Motion (ROM). Orthopedic repair or reconstructive surgery is still in its early stages of development. Presently, there is no viable technique to replace small bones in the wrist, foot, vertebrate or ankle to allow for normal ROM. The end goal of all implant surgery is to obtain permanent standard ROM for the affected area.

Currently joint implants and cartilage or ligament repair is accomplished by using “Off-the-Shelf” implants, (as whole units or assembled from a selection of parts), or donor implantable parts of specific sizes and strengths. In order to accommodate the many individuals that will receive them, such implants or repairs require a protracted period of time to be developed. Additionally, the implant then has to receive F.D.A. approval after trials of the implant have been conducted. For larger joint implants there exists modular components that can be assembled for a more correct fit. For small bones of the hand and foot, there are no implants available, with the exception of finger joints. Voxelizing geometric models of objects in 3D space allows for conversion of the 3D models into machine language that can be interpreted through the CNC process into physical models that are used in the development of the implants to test their efficacy before the first implantable prototype is realized. (See, for example, U.S. Pat. Nos. 5,995,738 and 6,556,199, the teachings of which are incorporated herein by reference in their entireties.)

There are many disadvantages and limitations associated with current techniques. For example, due to difficulties in making the implant fit properly in the available body space, it may be necessary to limit or cut off blood supply to certain parts of the body for an undesirable period of time. The surgical site is exposed, and therefore, there is an increased risk of infection while the surgical team re-works the implant and/or reduction and/or reshaping of living tissue to perfect the fit of the implant. After joint replacement surgery and the initial healing process, the implanted “off-the-shelf” replacement parts may cause secondary wear on other parts of the patient's body. This secondary wear occurs as the body tends to compensate for the inaccurate geometry of the implants. Due to stress imposed by the implant, minute restrictions or undesirable extensions may lead to degradation of secondary site-specific and non-site-specific biological tissue.

Thus, in order to reduce the amount of time needed for surgery, and to achieve a more exact fit, a need exists for improved processes of reproduction of hard and soft tissue implants. Such implants would include joint replacement parts, and the methods of preparing such implants and the biological tissue to which they are attached.

### SUMMARY OF THE INVENTION

It has now been discovered that, using technology derived from rapid prototyping of parts with CNC machining technology, a database can be constructed from a series of body imaging scans of the affected area in an individual, and an implant can be made specifically for the individual recipient, in a matter of days if necessary. This would reduce time scheduled for surgery as well, by reducing the amount of time required to ready the patient and implant for the actual implantation process, achieving a better fit that optimizes the correct anatomical geometry necessary for the body to function without compensating for the implant.

The invention inter alia includes the following, alone or in combination. In one embodiment, the invention relates to a system of preparing a one of a kind artificial biological implant to repair or replace a tissue part in a living individual, the system comprising: pre-operatively constructing an anatomically correct, affected area 7-unique scan implants geometry, wherein the implants geometry is unknown before the acquisition of the individual's data; and using a process derived from the combination of rapid prototyping technology and an existing FDA approved implant device to construct a geometrically and anatomically correct duplication of the functional attributes of the tissue or bone part to be replaced.

One embodiment of the invention is a custom-made implant to repair tissue or bone in areas of the body that heretofore have not received serious consideration from the medical community. Development of such implants has not yet occurred due to the potentially low return on investment. The implant prepared by the 3-D modeling system explained in the previous paragraph would construct a one-of-a-kind implant to fit into the individual with anatomical correctness. An example is a carpal bone, such as a Scafoid or Lunate, that would preserve the integrity of the wrist ROM with minimally invasive surgery, rather than removal of a row of carpas, proximal row carpectomy, or fusion. Existing wrist replacement technologies require removal of carpas and penetration or removal of the distal articular cartilage of the radius to implant them, thereby causing degradation of joint strength and ROM.

The present invention has many advantages. The implants and methods according to various embodiments of the invention facilitate tissue replacement surgery such as knee replacement surgery. A custom-made implant according to an embodiment of the invention can be constructed specifically for an individual recipient. If necessary, the custom-made implant can be produced according to the disclosed system in a matter of days. To the great advantage of the patient and surgeon, use of an embodiment of the invention reduces the time interval between the trauma to an area and the surgery. This increases the probability of success of the surgical repair. Use of an embodiment of the invention also helps avoid secondary wear on other parts of the body that are attempting to compensate for the inaccurate geometry that can arise from off-the-shelf, or “close match” implants. It also reduces open time at the site to minimize trauma and the chance for infection.

### BRIEF DESCRIPTION OF THE DRAWINGS

The foregoing and other objects, features and advantages of the invention will be apparent from the following more particular description of illustrative embodiments of the invention, as illustrated in the accompanying drawings in which like reference characters refer to the same parts throughout the different views. The drawings are not necessarily to scale, emphasis instead being placed upon illustrating the principles of the invention.

FIG. 1 (Prior Art) is an example of a cross sectional MRI and the layers at which scans were made.

FIG. 2 (Prior Art) is the full legend of the MRI depicted in FIG. 1, and includes scale, distance between scans, subject matter, precision, time and date and orientation.

FIG. 3 X-3 Z (Prior Art) shows the three axes scanned relative to 3-D computer space, X, Y, Z respectively.

FIG. 4 (Prior Art) depicts an assembly in 3-D modeling space of a part of each of the three intersecting scans on each plane to demonstrate placement.

FIG. 5 (Prior Art) depicts the resulting intersecting MRI images projected as background images in a CAD program registered at point 0,0,0, with each previous or post scan projected at the distance prescribed by the legend in FIGS. 1 and 2.

FIG. 6 X through FIG. 6Z (all Prior Art) depicts the ability to represent and construct the scanned area through voxelization of the entire database represented by all scans. FIG. 6X (Prior Art) depicts the result of an MRI scan of a joint. FIG. 6Y (Prior Art) represents a volumetric construction of the same joint through voxelization of the entire database represented by all scans. FIG. 6Z (Prior Art) represents a 3-dimensional view prepared by the data bases.

FIG. 7A is a perspective view of a schematic representation (30) of affected bone and ligament, and healthy ligament removed from its environment for illustration purposes.

FIG. 7B is a schematic representation (40) of parts of a disclosed implant embodiment and live tissue.

FIG. 7C is a schematic representation (60) of a disclosed implant embodiment and ligament connected at the implant site without surrounding tissue.

### DETAILED DESCRIPTION OF THE INVENTION

A description of preferred embodiments of the invention follows. It will be understood that the particular embodiments of the invention are shown by way of illustration and not as limitations of the invention. At the outset, the invention is described in its broadest overall aspects, with a more detailed description following. The features and other details of the compositions and methods of the invention will be further pointed out in the claims.

The present invention is directed to custom-made implants and methods of preparing a custom-made implant to repair tissue in a living individual. Table 1 summarizes steps in the disclosed system.

TABLE 1
Progression of process of development for patient specific implant.

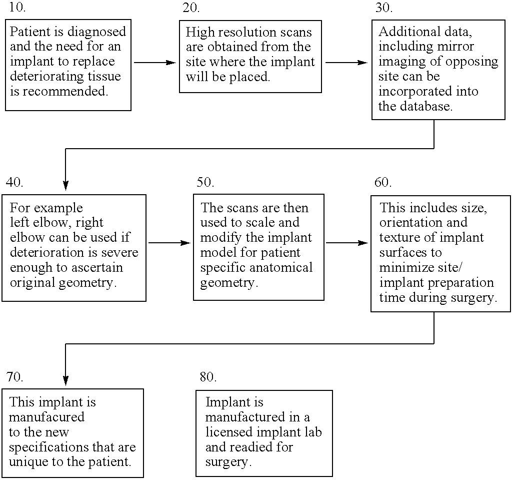

An implant, for example, a bone that is part of a complex joint, according to an embodiment of the invention, may have a structure that does not reflect the actual biological component it is designed to replace.

An example of a disclosed implant is a superstructure of artificially made components that has the correct surfaces in the anatomically correct positions, but may also have holes cut through the implant where ligaments are attached so that the ligaments pass through the implant, eliminating the necessity of having ligaments attach to the bone.

A disclosed implant may or may not require surfaces for tendons and/or ligaments to adhere to, because living tissue may pass through the implant with enough bearing surface on the living tissue as is necessary to maintain it's correct anatomical position.

A disclosed bone implant can be constructed of high density medical grade polymers entirely, or in combination with stainless steel or titanium, providing both the strength of bone and cushioning effect and lubricity of cartilage necessary to replace the biological components of bone and its articular cartilage.

A breakdown of the implant's components may be necessitated by the complexity of its ligament and tendon attachments.

A key component of one embodiment of a disclosed implant is a locking device that holds the implant together and allows it to be unlocked and removed from the interconnected tendons should it's replacement be necessary, or additional implants be required to attach to it due to a genetic defect in the individual. An example of such a genetic defect is Osteo-arthritis or a similar degenerative disorder.

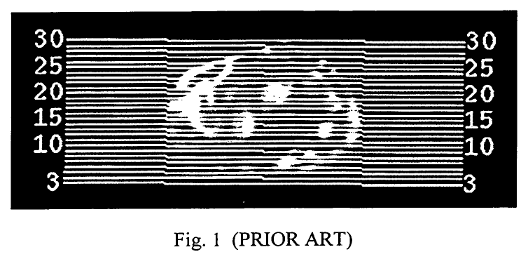

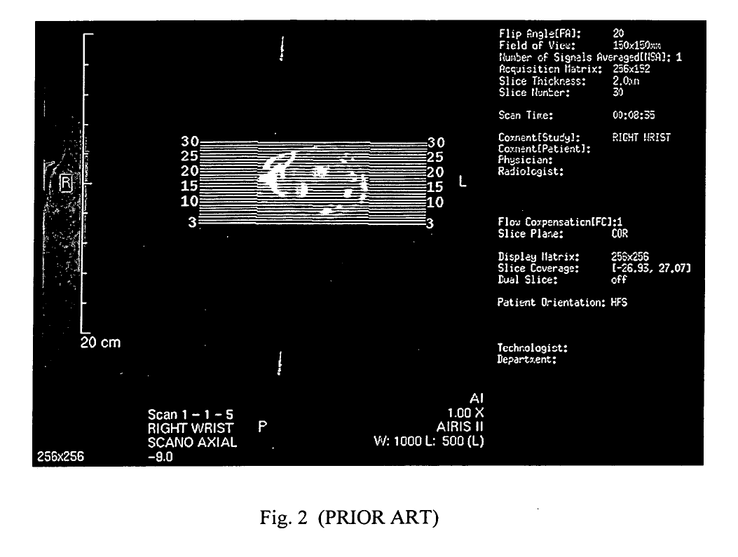

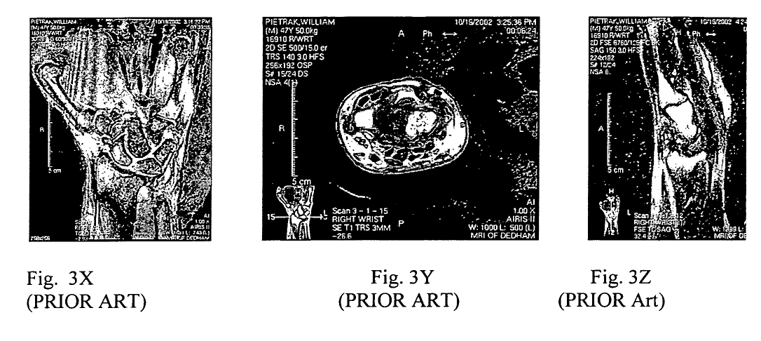

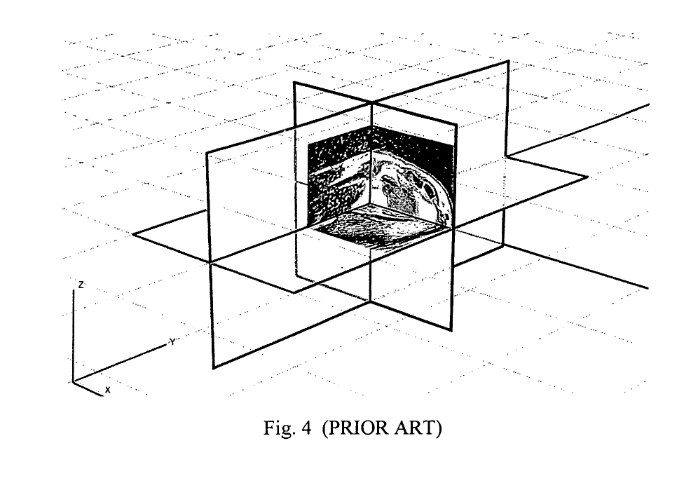

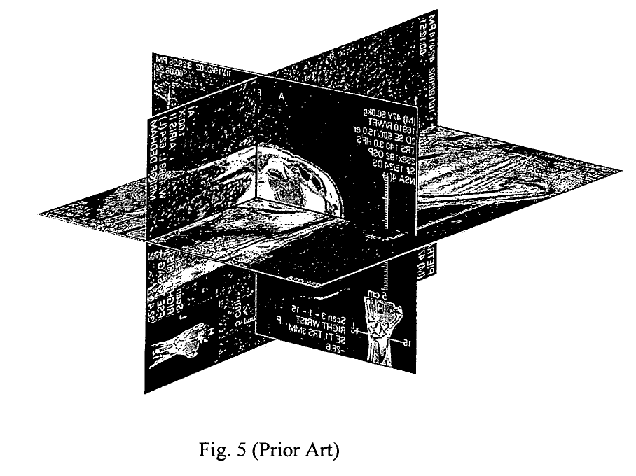

In one embodiment of the invention, a series of intersecting MRI images of a joint to be replaced or repaired are first obtained. The MRI images are then projected as background images in a CAD program registered at point 0,0,0, as shown in FIG. 5. Each previous or post scan would be projected at the distance prescribed by the legend in FIGS. 1 and 2. This method could be done by hand or computer program that discerns between the objects it is programmed to “see” to create outlines of, in this case bone and cartilage.

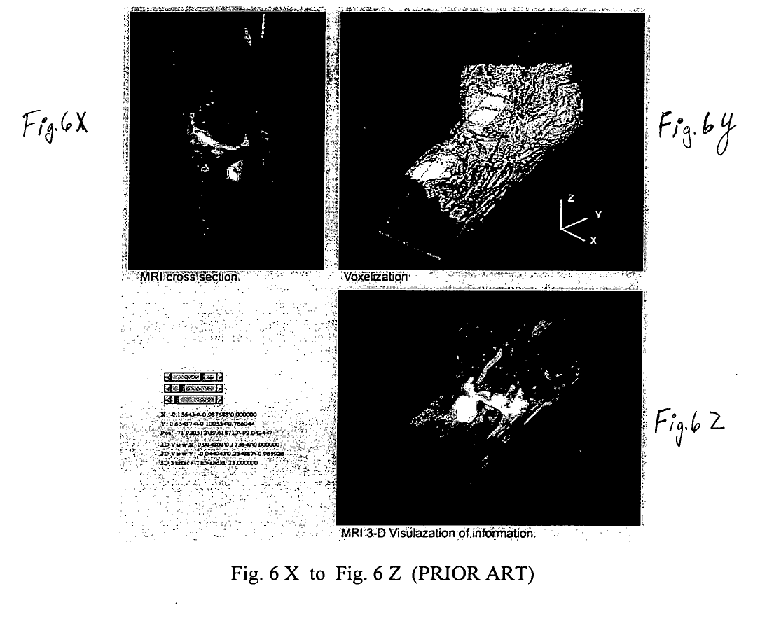

As shown in FIG. 6, through voxelization of the entire database represented by all scans of the target area, the scanned area is then represented or constructed in 3-D.

To this point these methods are being applied to reconstruct and insert the database for existing pre-surgery screening of parts to simulate, in this instance, the interplay between the database and the implant.

It has now been discovered that a custom-fit implant can be prepared with the existing database, and tested for continuity and fit. The implants database is then exported to a rapid prototyping program via machine language for Computer Numerical Control (CNC), a machine that automatically makes a part from a CAD program, machining, or exported through a file such as, for example, an STL file (a CAD file format for making Stereo Lithographic prototypes (SLA's)), to make a Stereo Lithographic solid that can be used in the lost wax process common in the casting of metal, or injection molding medical grade plastics. It is also advantageous that Non-uniform Rational B-Splines geometry (NURBS), rather than a Solids modeling program can be used to achieve organically and anatomically correct geometry, as these programs can output SLA files as well but are not limited by straight and curved line geometry.

Using an embodiment of the system or process of the invention, one can match a bone, for example, that has deteriorated in the left hand by arthritis. The first step is to scan the left hand and then the right. Being aware of the fact that bodies are not perfectly symmetrical, the data from the right hand can be a close approximation of the left. The stable relationship between the “good” hand can guide us in the reconstruction of the bone and articular cartilage necessary to repair the left hand.

Another embodiment of the system and a composite of the invention relates to the replacement of a broken vertebrate in the spinal column. Scanning the spine will provide the necessary measurements with which to make the replacement implant and, if necessary, the disks at the same time while the ability to make the implants in several parts would allow the implant vertebrate to be assembled around the spinal column.

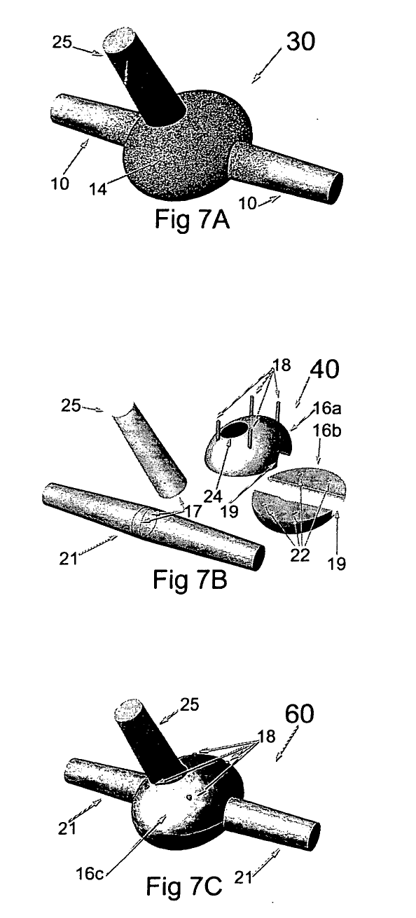

A schematic representation (30) of affected bone (14) and ligament (10) as well as healthy ligament (25) is shown in FIG. 7A.

FIG. 7B is a schematic representation (40) of parts (16a, 16b, 18, 21,25) of a disclosed implant, shown prior to assembly at a target implant site in an individual, and sections of live tissue such as donor ligament (21) needed for the repair. (17) Shows the surfaces of the ligaments where they would be attached inside the implant. Implant part 16A comprises a surface including a plurality of spaced fastener posts (18) positioned to align with the fastener post apertures (22) on part (16b). The fastener posts (18) on 16a) are positioned to align with the fastener post apertures (22) on (16b) and to lockingly engage the two parts upon insertion into a target tissue site. Implant part (16a, 16b) defines apertures and channels (19) through which ligament (21, 25) may be passed and connected at (17). The living tissue will be connected to other living tissue and these implants attached to correlating ligaments or attachment point of ligaments in the body. If there is enough living tissue (25) at the insertion site, donor tissue may not be necessary to complete the implant if the resident tissue is both healthy and of enough substance to complete the implant process.

FIG. 7C, a schematic representation (60) of a disclosed reconstructed bone and ligament, depicts implant part 16C, comprised of (16a, 16b) with ligament (25) inserted through aperture (24); and ligament (21) passed through aperture (19) shown in FIG. 7B. The ends of fastener posts (18) are shown protruding through implant part (16C). Protrusion of posts would not be normal for a final implant.

It should be noted that the schematic representations shown in FIGS. 7 A and C appear to be very similar to one another. The similarity is due to the fact that the implant will have attributes in function and form (limited by void left by degenerated tissue) that are virtually identical to those of the biological component it is replacing. Ligament is attached to ligament, thereby eliminating the need for surface attachments. Connection of the ligaments is dependent on the situation and is described herein. Articular cartridge and bone structure are not separated in FIG. 7C, because the exact arrangement would differ from implant to implant, and the optimal arrangement could be determined by those of skill in the related surgical art with no more than routine experimentation. It should also be noted that only FDA approved materials can be used in the process. Further, labs will have to be certified to make the implants.

### EQUIVALENTS

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of preparing a one of a kind artificial biological implant to repair or replace a tissue part in a living individual, the method comprising:
a) pre-operatively constructing an anatomically correct, affected area 7-unique scan implants geometry, wherein the implants geometry is unknown before the acquisition of the individual's data; and
b) using a process derived from rapid prototyping to construct a geometrically and anatomically correct duplication of the tissue part to be replaced, while allowing for variance in its internal form and still providing the original tissue's properties.

2. The method of claim 1, wherein the implant is constructed to restore natural, anatomically correct geometry of the tissue part in a recipient and to eliminate the need for pre-operative kinematic biological testing of an implant.

3. The method of claim 1, wherein the tissue is a joint.

4. The method of claim 1, wherein the implant comprises material of natural origin.

5. The method of claim 4, wherein the material of natural origin is a bone.

6. A custom-made implant prepared by the system of claim 1.

7. The custom-made implant of claim 6, the implant comprising a first part and a second part, the parts configured to be joined together by a snap-together, post-insertion fit.

8. The implant of claim 7, wherein the first part comprises a surface defining a plurality of fastener post apertures and the second part comprises a surface including a plurality of spaced fastener posts positioned to align with the fastener post apertures and to lockingly engage the first part with the second part.

9. The implant of claim 7, wherein the first part is lockingly engaged with the second part to form an engagement having sufficient resistance to tearing and to breaking so that the implant when assembled meets or exceeds the resistance of the replaced tissue part to tearing and to breaking, had the replaced tissue part been healthy.

10. The implant of claim 7, wherein the engagement has sufficient resistance to tearing and to breaking so that the implant will not come apart under a natural extension phase of a ligament.
